SEQUENCE LISTING (3 sequences)

SEQ ID NO: 1 - protein, 459 aa, Human
MERLRDVRERLQAWERAFRRQRGRRPSQDDVEAAPEETRALYREYRTLKRTTGQAGGGLRSSESLPAAAEEAPEPRCWGPHLNRAATKSPQPTPGRSRQGSVPDYGQRLKANLKGTLQAGPALGRRPWPLGRASSKASTPKPPGTGPVPSFAEKVSDEPPQLPEPQPRPGRLQHLQASLSQRLGSLDPGWLQRCHSEVPDFLGAPKACRPDLGSEESQLLIPGESAVLGPGAGSQGPEASAFQEVSIRVGSPQPSSSGGEKRRWNEEPWESPAQVQQESSQAGPPSEGAGAVAVEEDPPGEPVQAQPPQPCSSPSNPRYHGLSPSSQARAGKAEGTAPLHIFPRLARHDRGNYVRLNMKQKHYVRGRALRSRLLRKQAWKQKWRKKGECFGGGGATVTTKESCFLNEQFDHWAAQCPRPASEEDTDAVGPEPLVPSPQPVPEVPSLDPTVLPLYSLGPS

SEQ ID NO: 2 - protein, 409 aa, Human
GQLAETPAEVFQALEQLGHQAFRPGQERAVMRILSGISTLLVLPTGAGKSLCYQLPALLYSRRSPCLTLVVSPLLSLMDDQVSGLPPCLKAACIHSGMTRKQRESVLQKIRAAQVHVLMLTPEALVGAGGLPPAAQLPPVAFACIDEAHCLSQWSHNFRPCYLRVCKVLRERMGVHCFLGLTATATRRTASDVAQHLAVAEEPDLHGPAPVPTNLHLSVSMDRDTDQALLTLLQGKRFQNLDSIIIYCNRREDTERIAALLRTCLHAAWVPGSGGRAPKTTAEAYHAGMCSRERRRVQRAFMQGQLRVVVATVAFGMGLDRPDVRAVLHLGLPPSFESYVQAVGRAGRDGQPAHCHLFLQPQGEDLRELRRHVHADSTDFLAVKRLVQRVFPACTCTCTRPPSEQEGAV

SEQ ID NO: 3 - protein, 340 aa, Human
GGERPVPKYPPQEAEQLSHQAAPGPRRVCMGHERALPIQLTVQALDMPEEAIETLLCYLELHPHHWLELLATTYTHCRLNCPGGPAQLQALAHRCPPLAVCLAQQLPEDPGQGSSSVEFDMVKLVDSMGWELASVRRALCQLQWDHEPRTGVRRGTGVLVEFSELAFHLRSPGDLTAEEKDQICDFLYGRVQARERQALARLRRTFQAFHSVAFPSCGPCLEQQDEERSTRLKDLLGRYFEEEEGQEPGGMEDAQGPEPGQARLQDWEDQVRCDIRQFLSLRPEEKFSSRAVARIFHGIGSPCYPAQVYGQDRRFWRKYLHLSFHALVGLATEELLQVAR

(19) United States
(12) Patent Application Publication
(10) Pub. No.: US 2012/0122085 A1
(43) Pub. Date: May 17, 2012

(54) RECQL4/RECQL4 VARIANT-P53 COMPLEX FOR ALTERED MITOCHONDRIAL FUNCTION IN ROTHMUND-THOMSON SYNDROME

(75) Inventor: Sagar Sengupta, New Delhi (IN)

(73) Assignee: NATIONAL INSTUTUTE OF IMMUNOLOGY, New Delhi (IN)

(21) Appl. No.: 13/318,667

(22) PCT Filed: Nov. 8, 2010
(86) PCT No.: PCT/IB10/02834

§ 371 Date: Jan. 26, 2012

(30) Foreign Application Priority Data
Nov. 10, 2009 (IN) 2321/DEL/2009

(51) Int. Cl. C12Q 1/68

(52) U.S. Cl. 435/6.1

(57) ABSTRACT

The present invention discloses a functional interaction of the p53 with RECQL4/RECQL4 variant. The present invention further discloses co localization of RECQL4/RECQL4 variant and p53 complex in the mitochondrial nucleoids. The MLS at the N-terminus of RECQL4 that binds to Tom 20 receptor complex and transport RECQL4/RECQL4 variant-p53 complex into the mitochondria has been identified. Further the invention discloses measurement of intracellular ROS, identification of mtDNA mutations as a diagnostic tool for RTS and treatment with ascorbic acid for scavenging ROS and as treatment for RTS.

### FIELD OF INVENTION

The present invention is in the field of biotechnology.

### BACKGROUND AND PRIOR ART

Maintenance of genomic integrity is critical as its instability is implicated in development and progression of cancer and in the aging process. One such class of enzymes involved in maintaining genomic integrity is the RecQ helicases which are encoded in human by the BLM, WRN and RECQL4 genes. Mutations in these genes result in autosomal recessive disorders such as Bloom syndrome (BS), Werner's Syndrome (WS) and Rothmund-Thomson Syndrome (RTS). RECQL4 gene encodes a protein of 1208 amino acids with a centrally conserved helicase domain. Sequences in the N- and C-terminal regions of RECQL4 protein do not have any similarity to other members of the RecQ helicases, indicating their importance in mediating specific functions. RECQL4, the protein was shown to have both nuclear and cytoplasmic localization (Petkovic et al., 2005; Werner et al., 2006; Yin et al., 2004). p300-mediated acetylation of RECQL4 may regulate the trafficking between nucleus and cytoplasm (Dietschy et al., 2009). Interaction of tumor suppressor gene p53 physically and functionally with members of the RecQ family of DNA helicases has been established. p53 can bind to both BLM and WRN and attenuate their activity to unwind Holliday Junctions (HJ) in vitro (Yang et al., 2002). Endogenous BLM and p53 bind in presence of DNA damage (Sengupta et al., 2003). Inactivation of p53 in BS cells led to a significant increase in the frequency of sister chromatid exchange (SCE), compared to BS alone, indicating that p53 and BLM cooperatively affect homologous recombination (HR). p53 can repress RECQL4 transcription by regulating the promoter occupancy of both itself and SP 1 in concert with histone deacetylase (HDAC1) (Sengupta et al., 2005).

EP 0999273 discloses the human gene RECQ4 and the proteins encoded by it and the variants of it. Also disclosed are antibodies to the RECQL4 protein, kits for diagnosing disease associated with RECQL4, transgenic and knockout animals of the RECQL4 gene and probes to the RECQL4 gene.

WO02/068672 is drawn to antisense compounds, compositions and methods for modulating nuclear RECQL4 expression, particularly with oligonucleotides hybridizable with nucleic acids encoding RECQL4.

WO2006/109091 discloses the identification of RECQL4 deficient condition and a method of treating a RECQL4 deficient cancer condition in an individual with the administration of DNA replication inhibitor to the individual.

Studies have shown that cells exhibiting RTS condition unlike normal human fibroblasts, contain high levels of persistent reactive oxygen species (ROS) after DNA damage. It may be an indication of the mitochondrial dysfunction where there is disruption in the free radical scavenging enzymes, which in turn result in the cascade of reactions—from premature aging to neoplastic conversion of the normal cells into their cancerous counterpart. There are no conclusive studies on the functional interaction of RECQL4 and p53, although nuclear and cytoplasmic localization of both had been established.

The present invention thus aims:

Disclosing the functional interaction of the p53 with RECQL4.

Identification of RECQL4/RECQL4 variant and p53 complex in the mitochondrial nucleoids.

Identification of mitochondrial localization signal (MLS) at the N-terminus of RECQL4 that binds to Tom 40 receptor complex and transport RECQL4/RECQL4 variant-p53 complex into the mitochondria.

Measurement of intracellular ROS and mitochondrial DNA (mtDNA) mutations as a diagnostic tool for RTS.

Treatment with ascorbic acid for scavenging ROS as a treatment for RTS.

### OBJECT OF THE INVENTION

The object of the invention is the identification of RECQL4/RECQL4 variant and p53 complex in the mitochondrial nucleoids.

Another object is the identification of MLS at the N-terminus of RECQL4 that binds to Tom 40 receptor complex (consisting of Tom20 and Tom70) and transport RECQL4/RECQL4 variant-p53 complex into the mitochondria.

Yet another object of the invention is the measurement of intracellular ROS and mtDNA mutations as a diagnostic tool for RTS.

Yet another object is the use of ascorbic acid for scavenging ROS and as treatment for RTS.

### BRIEF DESCRIPTION OF FIGURES AND TABLES

FIG. 1(A-B) depicts in vitro GST pull down assays to determine RECQL4-p53 interaction.

FIG. 2(A-G) illustrates the interaction of various mutant RECQL4 fragments with p53.

FIG. 3(A-B) illustrates the validation of RECQL4-p53 interaction using Flag-tagged RECQL4 and GFP tagged full-length p53.

FIG. 4(A-G) are the confocal photomicrographs of the co localization of RECQL4 with p53, BrdU and other mitochondrial proteins.

FIG. 5(A-G) depicts the involvement of Tom20 receptor (a member of the Tom40 receptor complex) in the transport of p53-RECQL4 into the mitochondria.

FIG. 6(A-D) depicts various models for identification of mitochondrial localisation signal in RECQL4.

FIG. 6(E-F) illustrates the validation of the mitochondrial localisation signal in RECQL4 by studying the relative binding of the wildtype and mutant helicase with Tom20

FIG. 7 illustrates the helicase function of RECQL4 on different substrates and how p53 regulates that function.

FIG. 8 demonstrates how p53 and its down stream target genes (Bax and p21) are activated in absence of RECQL4 in RTS patients.

FIG. 9A illustrates the constitutive nuclear localization of p53 due absence of RECQL4 in RTS patients.

FIG. 9B shows how the viability of RTS patient cells are decreased when exposed to different types of DNA damage.

FIG. 9C determines the mechanism leading to the lack of spontaneous apoptosis in RTS patient cells.

FIG. 10A illustrates the altered mitochondrial ultrastructure in RTS patient cells.

FIG. 10(B-D) illustrates the change in the mitochondrial mass and level of reactive oxygen species in RTS patient eells after exposure to DNA damage.

FIG. 10E demonstrates that treatment of RTS patient cells with ascorbic acid post exposure to mitomycin C decreases the load of reactive oxygen species.

FIG. 11(A, B) illustrates the change in the levels of SOD1 and SOD2 in RTS patient cells relative to NHF.

FIG. 11C illustrates the increase in the metastatic potential of the RTS patient cells.

Table 1 summarizes of prediction for RECQL4 for mitochondrial localization signal by different web based tools.

Table 2 showing the pair potential score for the interaction between Tom20 receptor structure and the predicted mitochondrial localization sequence in RECQL4

Table 3 lists the mutations detected in the mitochondrial DNA of RECQL4 fibroblasts.

### DETAILED DESCRIPTION OF THE INVENTION

Accordingly the present invention thus discloses the functional interaction of the p53 with RECQL4/RECQL4 variant. The present invention further discloses co localization of RECQL4/RECQL4 variant and p53 complex in the mitochondrial nucleoids.

The invention further identifies MLS at the N-terminus of RECQL4 that binds to Tom 20 receptor complex and transport RECQL4/RECQL4 variant-p53 complex into the mitochondria.

Further the invention discloses measurement of intracellular ROS and identification of mtDNA mutations as a diagnostic tool for RTS.

Further the invention discloses that the treatment with ascorbic acid for scavenging ROS and as treatment for RTS.

Accordingly, the present invention provides a method of identification of functional RECQL4/RECQL4 variant-p53 complex wherein the said method comprising the steps of:

a) interaction of p53 and RECQL4/RECQL4 variant

b) import of RECQL4/RECQL4 variant and p53 complex through Tom 40 receptor complex.

c) import of RECQL4/RECQL4 variant and p53 complex through Tom 40 receptor complex via the MLS of RECQL4.

d) the localization of the p53-RECQL4/RECQL4 variant complex in the mitochondrial nucleoids.

Further the invention discloses the interaction of RECQL4 and p53 resulting in masking of the nuclear localization signal of both RECQL4 and p53.

The functional MLS of RECQL4 is located at the N-terminal region of RECQL4.

The said MLS of RECQL4 is located between the 13th and 18th peptide of the N-terminal region of RECQL4.

The invention further discloses the lack of RECQL4 in RTS patients lead to deregulated mitochondrial functions.

Also, the invention discloses increased mtDNA mutations and enhanced metastatis of RTS fibroblasts with the loss of both RECQL4 and p53.

In another embodiment, the invention discloses a lentivirally expressed mitochondrially targeted RECQL4/RECQL4 variants which has the minimal interacting region for p53 and the helicase domain and can be used a vehicle to correct mitochondrial dysfunction. The “rescued” patient cells are then scored for the mitochondrial functions.

### EXAMPLES

The following examples are for the purpose of illustration of the invention and are not intended in any way to limit the scope of the invention.

### Example 1

Interaction between p53 and RECQL4 lead to the masking of their Nuclear Localization Signal

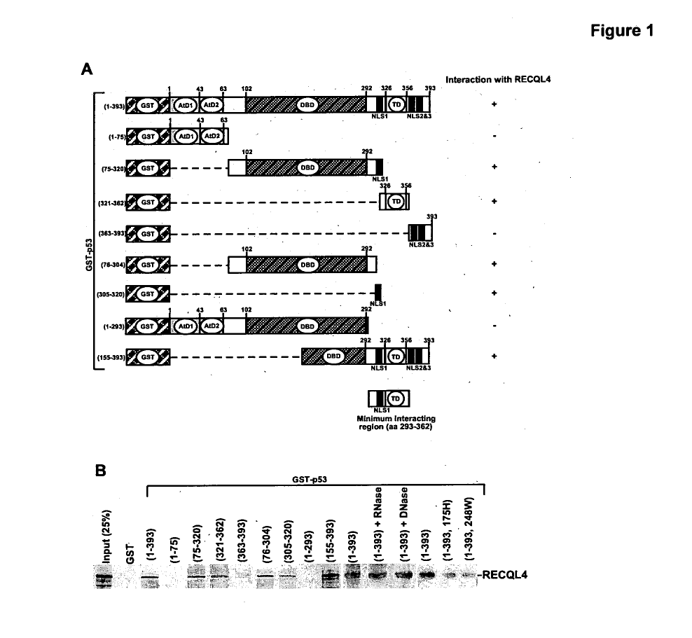

To determine RECQL4 interaction with p53 an in vitro GST pull-down assays with S35 labeled RECQL4 and the different deletion fragment of the tumor suppressor (FIG. 1A, 1B) was carried out. It was found that the minimal region of p53 that interacts with RECQL4 encompasses the amino acids 293-362. This region contains the major Nuclear Localization Signal (NLS1) of p53 along with the tetramerization domain (TD). p53-RECQL4 interaction is independent of nucleic acids. Two tumor derived mutants of p53 (175H, 248W), also show interactions with RECQL4, albeit to a reduced level compared to the wild-type tumor suppressor.

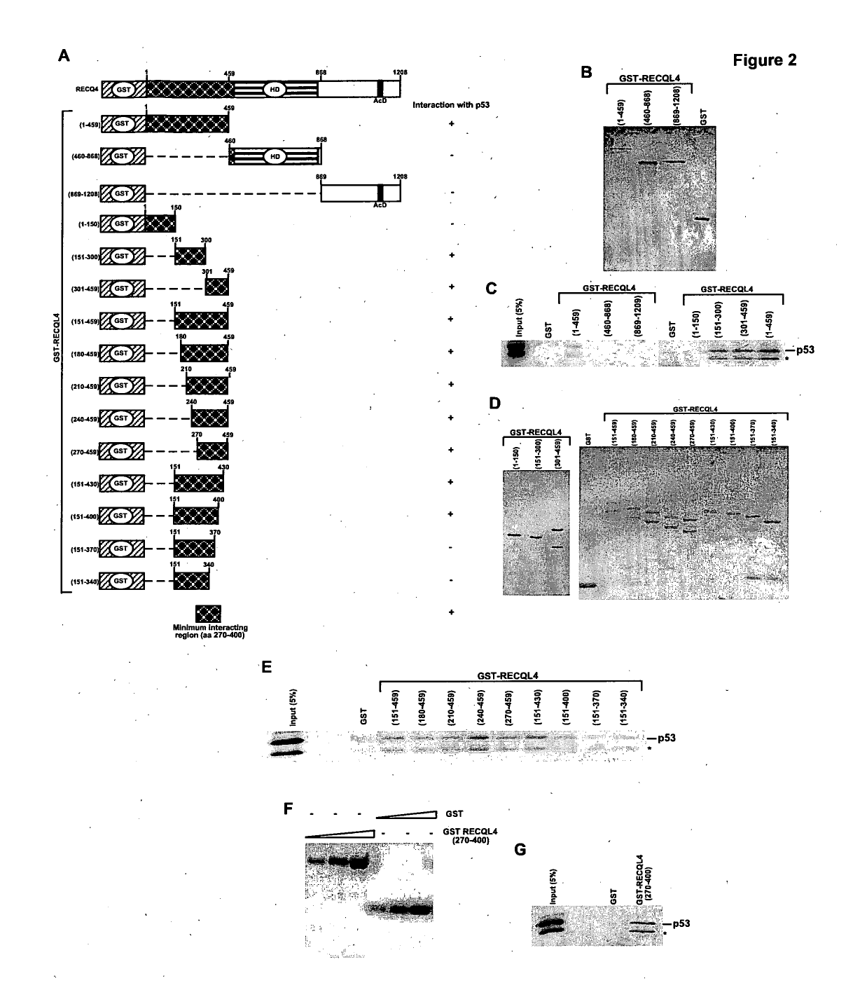

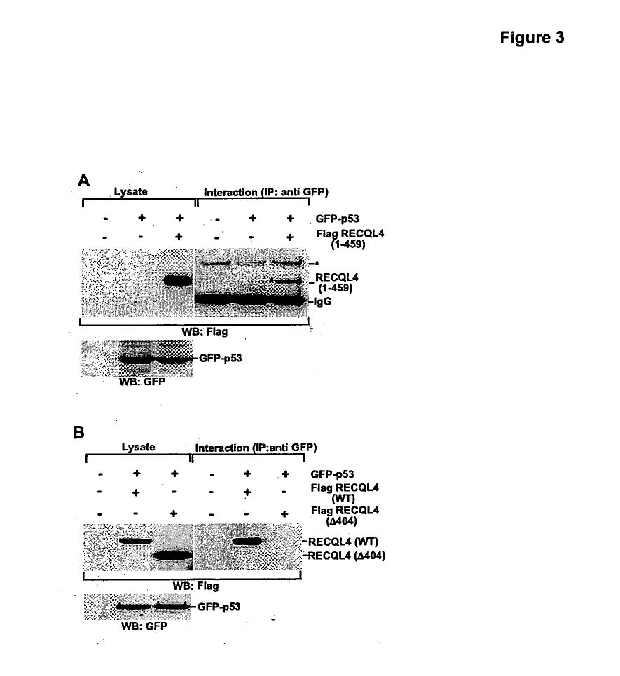

To determine the region of RECQL4, interaction with p53 three mutant protein fragments of RECQL4: 1-459, 460-868, 869-1208 (SEQ ID 1, 2 and 3) were generated and purified. The N-terminal 459 amino acids of RECQL4 interacted with p53 (FIG. 2A, 2B, 2C). Subsequently, N- and C-terminal mutants were generated within the first 459 amino acids, purified and GST pull down assays with S35 labeled p53 were performed and a minimal stretch of 130 amino acids (270-400) of RECQL4 interacted with p53 (FIG. 2D, 2E). The interaction with p53 was verified either with a GST-tagged RECQL4 (270-400) fragment in vitro (FIG. 2F, 2F) or by using a Flag-tagged RECQL4 (1-459) fragment in vivo (FIG. 3A). Further it was found that full-length RECQL4 but not the N-terminal 404 amino acid truncated variant interacted with GFP tagged full-length p53, thereby validating the region of interaction in the context of full length proteins (FIG. 3B).

### Example 2

RECQL4 and p53 Colocalize in Mitochondria:

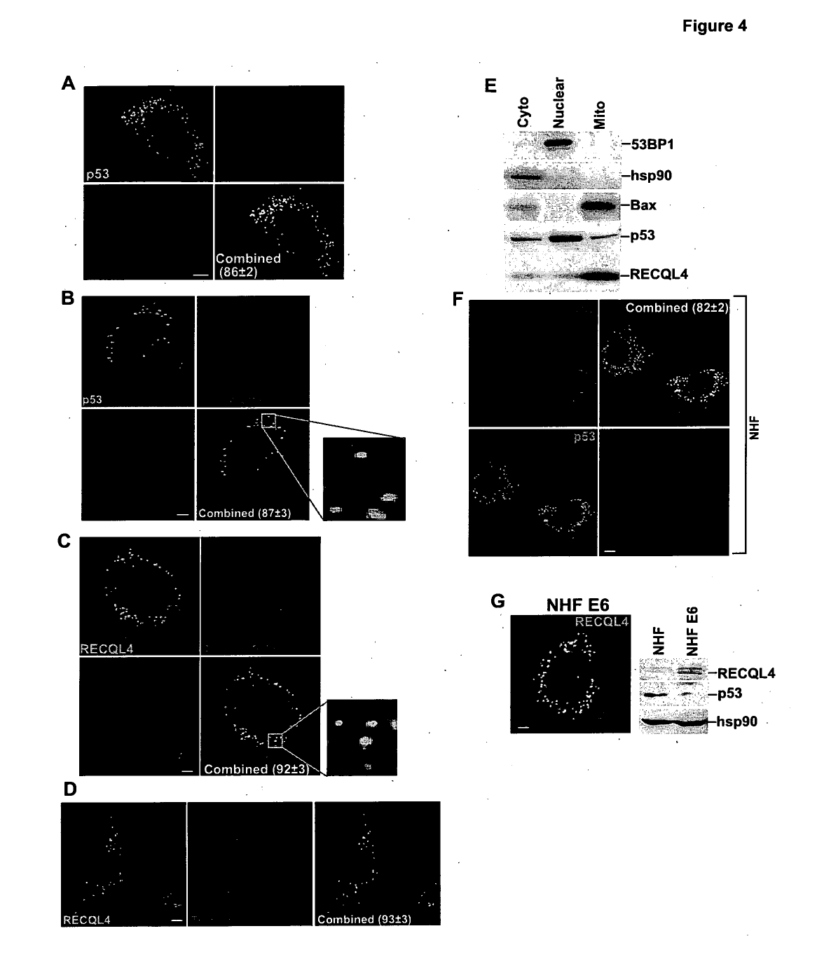

To determine the subcellular distribution of p53 and RECQL4/RECQL4 variant, we stained asynchronously grown NHF with p53 and RECQL4 antibodies and imaging with confocal microscopy was carried out. For confocal microscopy, the slides were analyzed on a Zeiss 510 Meta system with 63×/1.4 oil immersion or 40×10.95 Con objective. The laser lines used were Argon 458/477/488/514 nm (for FITC), DPSS 561 nm (for Texas Red and Mitotracker dyes) and a Chameleon Ultra autotunable femtosecond laser with a tuning range 690-1050 nm (for DAPI). LSM5 software was used for image acquisition. p53 and RECQL4 colocalized to a high degree in the cytoplasm as punctate staining (FIG. 4A) and also found that both p53 and RECQL4 colocalized extensively with mitochondrial marker, cytochrome c and Mitotracker Red (FIG. 4B, 4C) and mitochondrial helicase Twinkle (FIG. 4D). The mitochondrial localization of RECQL4 and p53 was further demonstrated by cell fractionation experiment (FIG. 4E). The integrity of the cell fractionation was tested by specific markers. Substantial amount of both p53 and RECQL4 were found to be present in nucleus and mitochondria. Since nucleoids contain mtDNA, we performed immuno-colocalization of RECQL4 and p53 with BrdU or Ethidium Bromide (EtBr) according to published protocols (Davis and Clayton 1996; Spelbrink et al. 2001). Both p53 and/or RECQL4 colocalized with BrdU (FIG. 4F). Using NHF E6 cells the localization of RECQL4 with mtDNA was found to be independent of p53 (FIG. 4G).

### Example 3

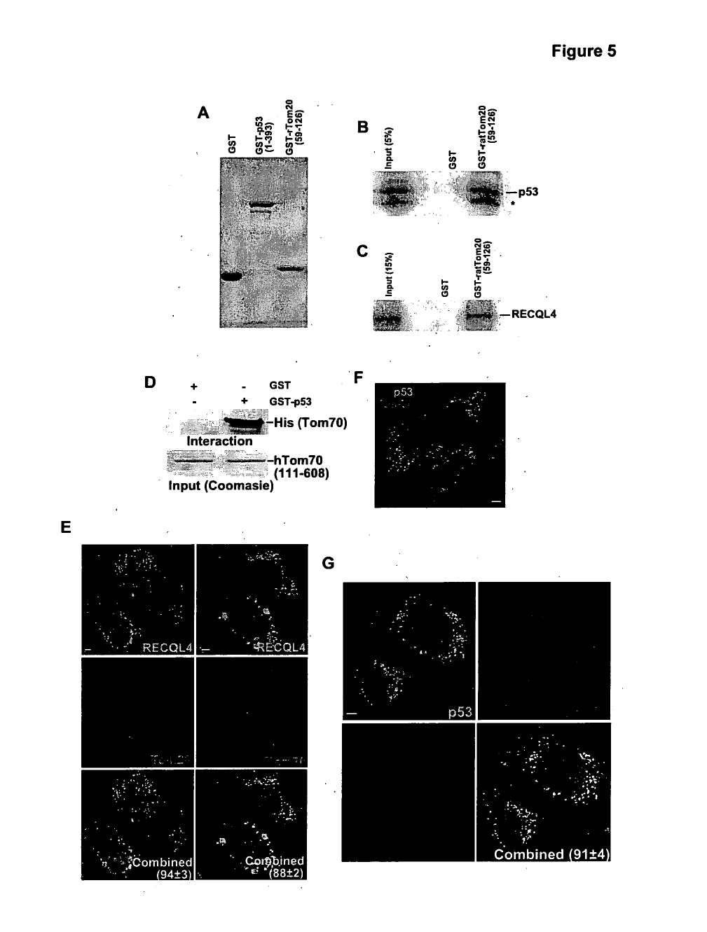

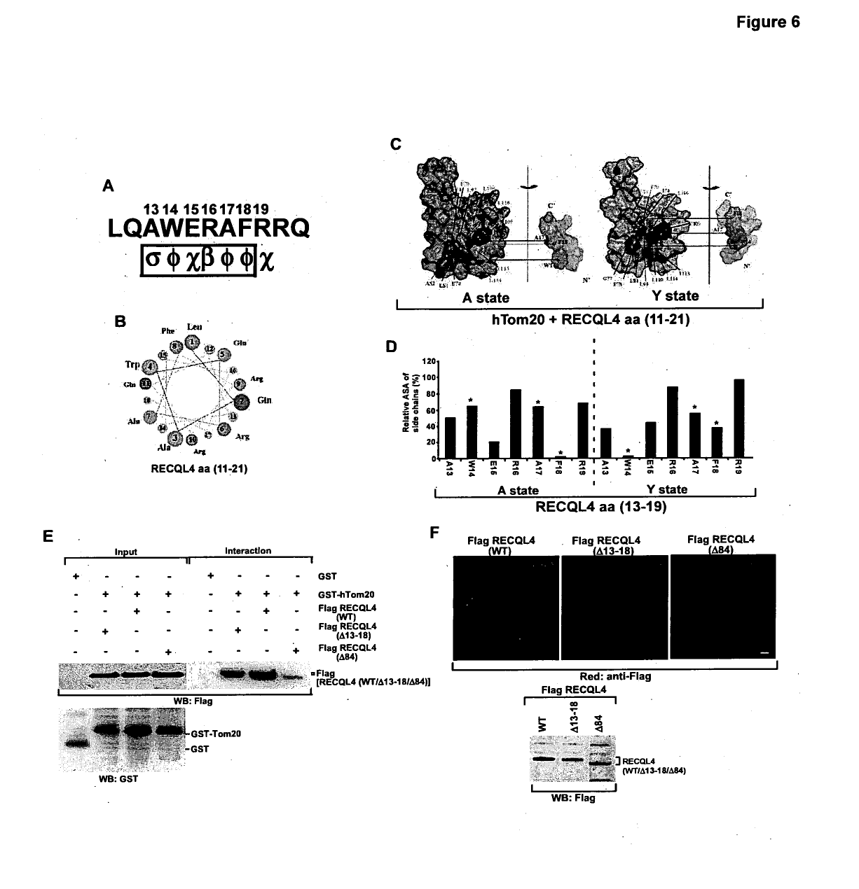

The MLS of RECQL4 was determined in the N-terminal region of the helicase: Since both p53 and RECQL4 were localized in the mitochondria (FIG. 4) the interaction with mitochondrial receptor proteins was studied. Using S35-labeled RECQL4 and p53 we found that both the proteins interacted in vitro with cytosolic part of the Tom20 receptor (FIG. 5A, 5B, 5C). p53 also interacted with the cytosolic part of Tom70 (FIG. 5D). Both p53 and RECQL4 colocalized extensively with Tom20 and Tom70 (FIG. 5E, 5F, 5G). Using four web based tools Target P, PSORT, MitoProt and iPSORT it was found that mitochondrial localization was predicted with high reliability and probability for RECQL4 especially the N-terminal domain (Table 1). We reasoned that the putative MLS of RECQL4 should be recognized by Tom20. A systematic analysis with the disulfide-tethering peptide library had been carried out to reveal the amino acid residues of the substrates involved in the interaction with Tom20 (Obita et al., 2003). The deduced motif was “σφχβφφχ” where φ, Hydrophobic; σ, hydrophilic; β, basic; χ any amino acid. We searched the N-terminal 100 amino acids of RECQL4 using the above motif. Interestingly, one sequence stretch ‘13AWERAF18” which matched with the defined motif (FIG. 6A) was found. The deduced pre-sequence was modeled onto the available solution structure [10M2.pdbj and the A state and Y state crystal structures of the rat Tom20 receptor [2VIT.pdb, 2VIS.pdbj (Saitoh et al., 2007) and binding affinity of this pre-sequence peptide with the Tom20 receptors was evaluated using a residue based statistical pair potential, namely the BT matrix (Betancourt and Thirumalai, 1999). Binding energies based on pair potentials gives us a score to distinguish between probable mitochondrial pre-sequence and non binders (Table 2), where we have compared the scores for the RECQL4 pre-sequence with the aldehyde dehydrogenase pre-sequence and a NLS in DNA helicase enzyme. The helical wheel plot also revealed that hydrophobic amino acids were concentrated on one side of the helix (which presumably faced the cytosol) while the hydrophilic amino acids were on the other side (which could bind to Tom20 receptor) (FIG. 6B), indicating that the predicted pre sequence is the most probable MLS. To have a structural overview of RECQL4-Tom20 interaction, all atom models of the predicted pre-sequence in RECQL4 were built in complex with Tom20 using SCWRL (Canutescu et al., 2003). The models generated by SCWRL were further energy minimized using the discover module of Insight II. Co-crystal structure studies have revealed that Tom20 in solution can bind to atleast two bound states of the presequence (Saitoh et al., 2007). The two states of the presequence (A state and Y state) were obtained by the usage of different linker designs. The open-book representation of minimized structures for the Tom20 receptor-RECQL4 presequence for both states 10 revealed the importance of Trp 14, Ala 17 and Phe 18 of RECQL4 in the binding (FIG. 6C). These residues form a hydrophobic surface of the amphiphilic ct-helix that interacts with the hydrophobic patch in the Tom20 groove. Two out of the three hydrophobic residues of the pre-sequence were tightly buried in the two different states of Tom20 receptor. This was further correlated by the relative surface accessibility of the side chain atoms of these hydrophobic residues in the modeled complexes of the A state and the Y state crystal structure of Tom20 receptor (FIG. 6D). Overall the in silico experiments predicted the presence of a mitochondrial localization sequence in the extreme N-terminal region of RECQL4, which could bind to mitochondrial receptor, Tom20. We wanted to determine whether the predicted mitochondrial presequence in RECQL4 actually affected its binding and localization of RECQL4 in vivo. For this purpose we generated two internal deletion mutants of Flag-tagged RECQL4: Δ13-18 and Δ84. We found that while there was a reproducible 15-20% decrease in the binding between RECQL4 and Tom20 in the Δ13-18 mutant, the binding was completely abolished in the Δ84 mutant (FIG. 6E). This result indicated that additional amino acid residues in the N-terminus of RECQL4 apart from residues 13-18 were required Tom20-RECQL4 interaction. However, unlike wild-type protein, both Δ13-18 and Δ84 RECQL4 mutants colocalized to nucleus to a high extent (FIG. 6F), indicating that amino acid residues 13-18 have the capability to act as the MLS for RECQL4 in vivo.

TABLE 1
Summary of prediction for RECQL4 for mitochondrial localization signal
by the four different web based tools.

| S. No | Name of the Tool | Prediction |
|---|---|---|
| 1 | TARGET P | P^{b} = 0.82 and RC^{a} = 2 (Complete RecQL4) |
| | | P^{b} = 0.82 and RC^{a} = 2 (RECQL4 1-140) |
| 2 | PSORT | P^{b} = 0.804 (RECQL4 1-140) |
| 3 | MitoProf | P^{b} = 0.953 (RECQL4 1-60) |
| 4 | iPSORT | RecQL4 as a mitochondrial localization protein |
| | | (RECQL4 1-30) |

Note:
^{a}RC is a measure of the size of the difference (‘diff’) between the highest and the second highest output scores. There are 5 reliability classes, defined as follows: 1: diff > 0.800 2: 0.800 > diff > 0.600 3: 0.600 > diff > 0.400 4: 0.400 > diff > 0.200 5: 0.200 > diff
^{b}P = Probability

TABLE 2
Pair potential score for the interaction between Tom20 receptor structure
(both NMR and X-ray crystal structure) and the predicted mitochondrial
localization sequence in RECQL4, rat ALDH pre-sequence and nuclear
localization signal of DNA helicase enzyme.
[1] NMR structure

| | | | Pair potential score for the |
|---|---|---|---|
| S. no | Model | Presequence | motif |
| 1 | RECQL4 | LQAWERAFRRQ | −9.09 |
| | (13-19) | | |
| 2 | Rat ALDH | GPRLSRLLSYA | −10.65 |
| | (14-20) | | |
| 3 | DNA helicase | SKNTGAKKRKI | +1.05 |
| | (638-644) | | |

[2] X-Ray structure (A state)

| | | | Pair potential score for the |
|---|---|---|---|
| S. no | Model | Presequence | motif |
| 1 | RECQL4 | LQAWERAFRAAG | −6.1 |
| | (13-19) | | |
| 2 | Rat ALDH | GPRLSRLLSAAG | −3.45 |
| | (14-20) | | |
| 3 | DNA helicase | NTGAKKRKIAAG | −1.59 |
| | (638-644) | | |

[3] X-Ray structure (Y state)

| | | | Pair potential score for the |
|---|---|---|---|
| S. no | Model | Presequence | motif |
| 1 | RECQL4 | LQAWERAFRYAG | −6.54 |
| | (13-19) | | |
| 2 | Rat ALDH | GPRLSRLLSYAG | −8.23 |
| | (14-20) | | |
| 3 | DNA helicase | NTGAKKRKIYAG | −0.74 |
| | (638-644) | | |

Rat. ALDH (14-20) is the positive control, while DNA helicase (638-644) acts as a negative ontrol

### Example 4

RECQL4 Helicase Activity is Inhibited by p53

RECQL4 helicase assays were carried out as for Twinkle (Korhonen et al., 2003). The two DNA substrates tested were prepared using the oligoes (listed below) and subsequently PAGE purified.

Oligo 1: 5′ GCA TGT CAA AAT ACC TGA ACT GCC GTG ATC ACC AAT GCA
GAT TGA CGA ACC TTT GCC CAC GTC GTC CAG GAA CTC CTC AG 3′
Oligo 2: 5′ GCT CCA CAG CAG GCC CAT GCA GTA GGC CTA GCA TGA CAA
TCT GCA TTG GTG ATC ACG GCA GTT CAG GTA TTT TGA CAT GC 3′
Oligo 3: 5′ CTG AGG AGT TCC TGG ACG ACG TGG GCA AAG GTT CGT CAA
T 3′

Oligo 1, Oligo 2 and Oligo 3 used for making 5’ single stranded tail substrate, while Oligo 1 and Oligo 2 used for making splayed arm substrate.

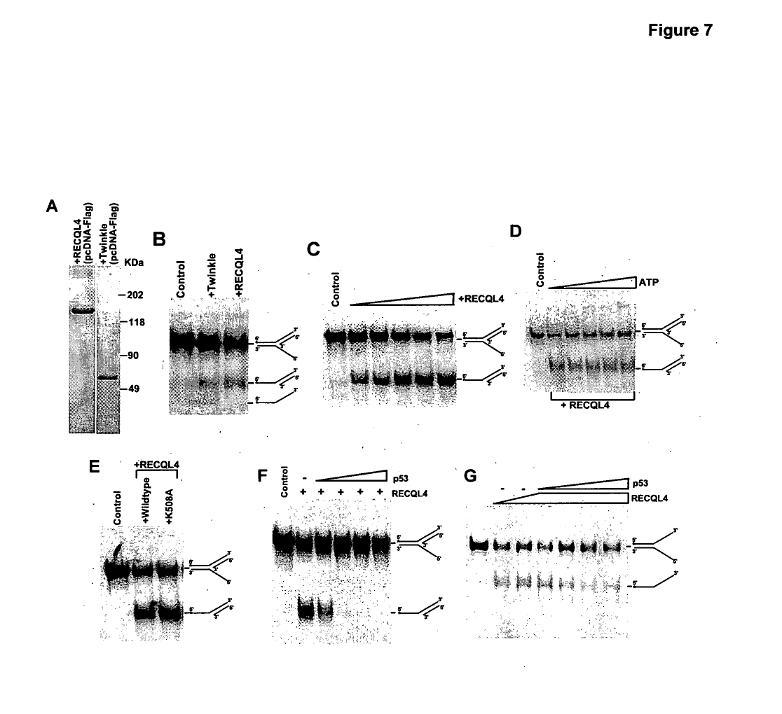

Using splayed-arm, blunt DNA and overhang substrates, it was concluded that RECQL4 had a 3′ to 5′ helicase activity (Xu and Liu, 2009) (which we have confirmed, data not shown). However, on longer incubations, a 5′ to 3′ helicase activity for RECQL4 was detected, much like E. coli RecQ helicase (Umezu et al., 1990). We reasoned that if RECQL4 was a “bona fide” mitochondrial helicase it should also unwind similar substrate(s) as Twinkle (Korhonen et al., 2003). Purified Flag-tagged full-length RECQL4 and Twinkle (FIG. 7A) could both unwind 5′-single stranded tail substrate to similar extent (FIG. 7B). The unwinding activity of RECQL4 was enhanced in a protein and ATP concentration dependent manner (FIG. 7C, 7D). As reported (Xu and Liu, 2009), RECQL4 mutant K508A displayed robust unwinding activity, confirming that the second ATP binding region in the N-terminus contributed to the 5′ to 3′ helicase activity (FIG. 7E). Like for BLM and WRN (Yang et al., 2002), p53 inhibited RECQL4 unwinding activity on 5′ single stranded tail (FIG. 7F) and splayed arm substrates (FIG. 7G).

### Example 5

Determination of the Consequence of Loss of RECQL4 in Cells from RTS Patients:

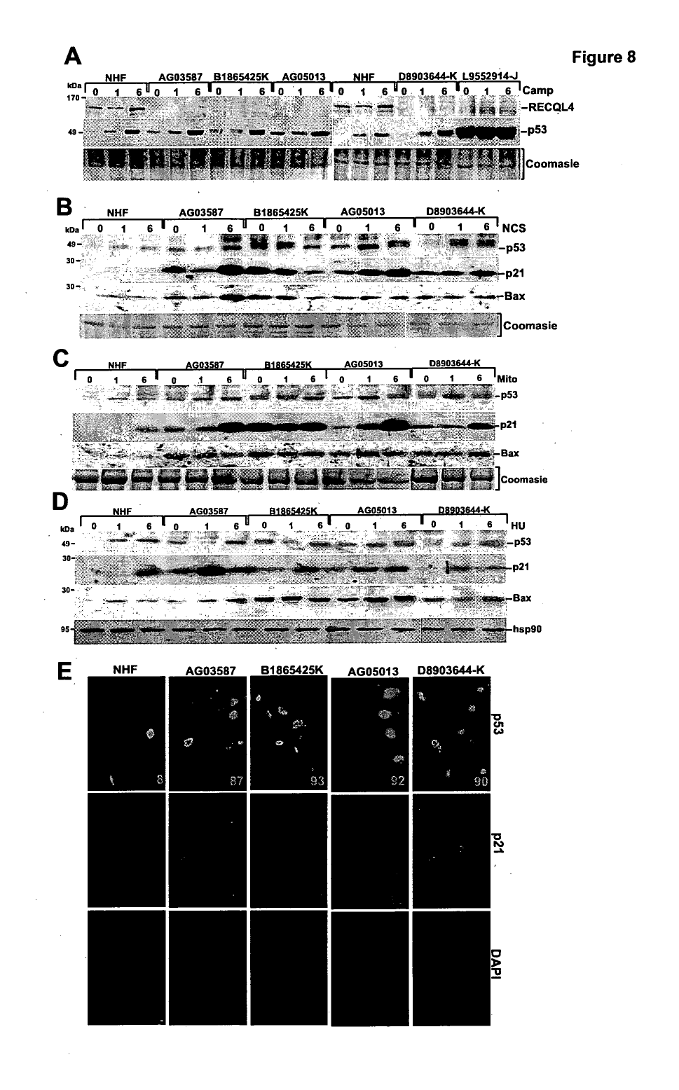

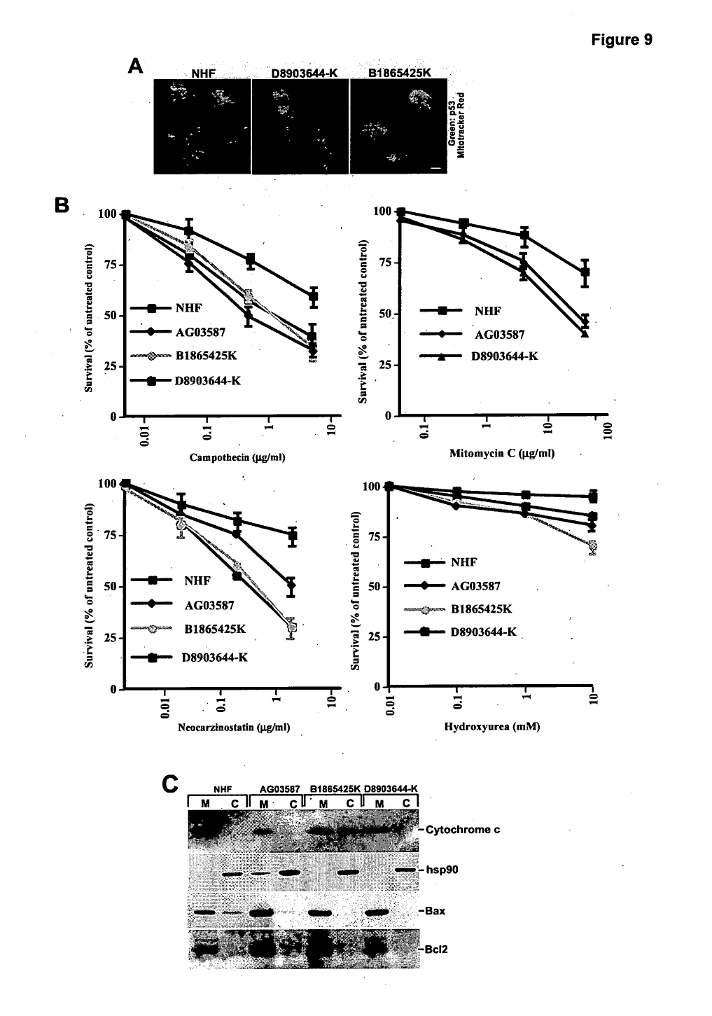

For this purpose patient fibroblasts characterized by us (Kitao et al., 1999; Lindor et al., 2000) or available from Coriell Institute of Medical Research were used. To facilitate long-term studies, the cell lines were immortalized using hTERT. Lack of RECQL4, as confirmed in immortalized RTS cells, led to the accumulation and activation of transcriptionally active nuclear p53 and thereby its downstream target genes like p21 and Bax (FIG. 8). Consequently very little of the p53 was found in the mitochondrial nucleoids in the RTS fibroblasts (FIG. 9A). Compared to NHF, p53 in the RTS cells was elevated to a much higher level after NCS, HU, CPT or Mito treatment. Consequently levels of p53 targets, p21 and Bax were also elevated (FIG. 8). These results indicated that while p53 could not independently go to the mitochondria in absence of RECQL4, its interaction with RECQL4 in the mitochondrial nucleoids was necessary to prevent its spontaneous upregulation and conversion into a transcriptionally active form in the nucleus. We next performed long-term clonogenic assays with NHF and RTS fibroblasts. We found that the survival fraction of all the four RTS fibroblasts tested was less (compared to NHF) after DNA damage (FIG. 9B). These results indicated that the lack of RECQL4 coupled with abnormal nuclear accumulation of p53 sensitized the cells towards cell death induced by different types of DNA damage. Next we wanted to determine why despite enhanced p53 level, RTS cells did not undergo spontaneous apoptosis. Hence we determined the amount of intracellular cytochrome c, the mitochondrial fractions (prepared by Mitochondria Isolation Kit for Cultured Cells, Pierce) were lysed on ice according to manufacturer's protocol. Post lysis, western analysis was carried out to determine the relative levels of cytochrome c in NHF vs RTS cell lines. Decrease in level of cytochrome c, but no change in bax/bcl2 ratio indicated a possible reason for the lack of spontaneous apoptosis in RTS patient cells (FIG. 9C).

### Example 6

Determination of the Compromised Mitochondrial Functions in the RTS Cells Grown Under Asynchronous Conditions:

For ultrastructural studies cell pellet was obtained after scrapping and centrifugation of the cultured cells followed by fixation in 4% paraformadehyde and 3% glutaraldehyde fixative in phosphate buffer (pH 7.2) for 24 hr and subjected to routine transmission electron microscopy (TEM). Briefly the cells were post fixed in 1% osmium tetroxide for 1 hr, followed by dehydration in grades of ethyl alcohol and cleared in propylene oxide, later embedded in Araldite CY212 resin and polymerized at 60° C. in oven for 48 hours. The blocks were cut in Leica EM UC6 ultramicrotome (M/S Leica Mikrosysteme, Austria). After initial screening, several ultrathin (400-500 A°) sections were collected on copper grids and stained with Uranyl acetate and Lead citrate as described by (Frasca and Parks, 1965). Sections were scanned under Tecnai 02 Spirit transmission electron microscope (FET Netherlands) and interested areas were photographed using Megaview-IJI digital CCD camera at 80 KVA.

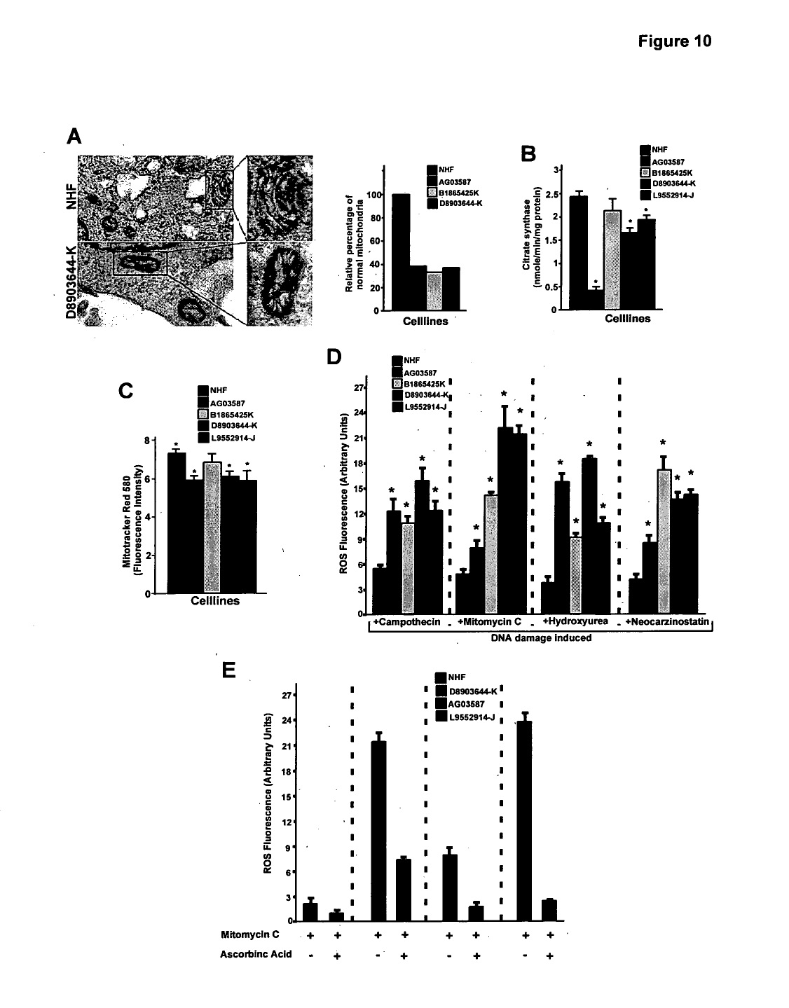

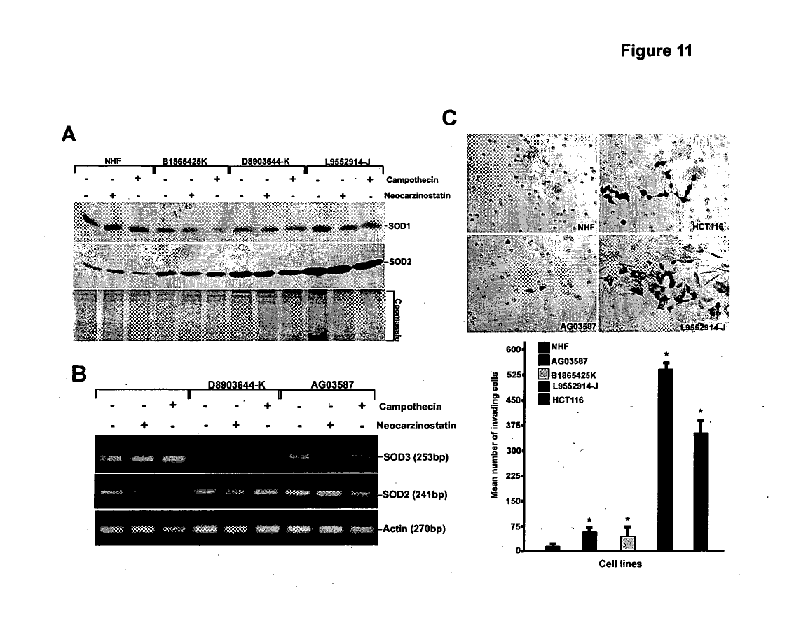

By transmission electron microscopy (TEM) we observed that the mitochondrial cristae were distorted and the intercristal space expanded in all the RTS fibroblasts grown under asynchronous conditions (FIG. 10A). The extent of the cristae space is known to affect the overall chemiosmotic function in the cell (Mannella, 2006). Since chemiosmosis is an indicator of electron transport chain (Mitchell, 1961), the TEM data indicate that the production of ATP is deficient in RTS patients not exposed to any DNA damage. Apart from ATP, mitochondria also produce the reactive oxygen species (ROS). Spontaneous ROS levels are usually low in normal cells as elevation of ROS levels can result in significant damage to cell structure (Schumacker, 2006). Indeed the level of ROS in asynchronous NHF was low as determined by the oxidation of 5-(and-6)-chloromethyl-2′,7′-dichlorodihydrofluorescein diacetate, acetyl ester (CM-H2DCFDA). Interestingly all the RTS fibroblasts show a reduced level of spontaneous ROS. Citrate synthase activity, considered a biomarker for mitochondrial mass and function was decreased in all RTS fibroblasts compared to NHF (FIG. 10B). Mitochondrial mass directly measured by Mitotracker Red 580 also showed the same trend (FIG. 10C). Mitochondrial mass was determined by usage of a fluorescent dye Mitotracker Red 580 (200 nM, Invitrogen). Live cells were incubated with of this dye for 30 min. The cells were subsequently washed, fixed and then analyzed in a Zeiss 510 Meta system with a 40×/0.95 Con objective. The fluorescence was captured initially for NHF and the acquisition parameters were kept unchanged for the capture of the fluorescence for the RTS cells. Biochemical determination of mitochondrial mass was performed by determining the citrate synthase activity in whole cell extracts using a kit (Sigma), following manufacturers' protocol. The activity was measured at 412 nm at 30° C. using 10 μg for each sample. Activity was expressed as nmole/min/mg of protein. Intracellular ROS in RTS cells was measured by the oxidation of CM-H2DCFDA (4 μM, Invitrogen). Live cells, grown on chambered slides (Nunc), were stained with a fluorescent CM-H2DCFDA for 30 mm in phenol red free medium (without serum) at room temperature. Cells were washed, and the fluorescence was captured in a Zeiss 510 Meta system with, a 20×/0.8 objective. The laser line used was Argon 458/477/488/514 nm. LSM5 software was used for image acquisition. The fluorescence was captured first for NHF and the acquisition parameters were kept unchanged for the subsequent capture of the fluorescence for the RTS cells. For the DNA damage experiments, the cells were treated with the respective drugs for 6 h. The cells were subsequently washed and the incubation continued for a further 18 h before the fluorescence readings were carried out as above. We found that the RTS cells contained abnormally high levels of ROS, 18 h after exposure to four different types of DNA damage (FIG. 10D). ROS scavengers like ascorbic acid reversed this effect (FIG. 10E). Expression levels of the major superoxide scavenger enzyme active in the mitochondria, superoxide dismutase 1 (SOD1) and the enzyme secreted into extracellular space, superoxide dismutase 3 (SOD3) were both decreased at protein and RNA level (FIG. 11A, 11B).

### Example 7

mtDNA Mutations Accumulate in RTS Fibroblasts:

mtDNA is especially susceptible to ROS as its location is in close proximity to the sites of ROS production from the respiratory chain, apart from lack of protection by nucleosomes and limited DNA repair capabilities of mitochondria. Mutations in mtDNA have been reported in a variety of cancers (Modica-Napolitano et al., 2007). We hypothesized that high level of ROS in RTS fibroblasts could lead to accumulation of mtDNA mutations. We carried out complete mitochondrial genome sequencing of NHF and three RTS, patient celllines (AG03587, B1865425K and L9552914-J). Sequencing of the entire mitochondrial genome was carried out by using the mitoSEQrTM System. This system allows identification of sequence variation in the entire human mitochondrial genome and its control regions. Overlapping region in the mitochondrial genome was amplified with specific primer pairs tailed with universal M13 sequences to generate resequencing amplicons

(RSAs). The RSAs were then used as templates for sequencing using universal primers. The entire process was carried out with mitoALLTM kit (Applied Biosystem) in an Applied Biosytem 3130 Genetic Analyzer. The data was analyzed using SeqScape software v2.6. Every mutation reported was verified by duplicate analysis. Comparison with revised Cambridge Reference Sequence revealed the listed polymorphisms in NHF and RTS cells (data not shown). The three RTS fibroblasts (but not NHF) accumulated additional mutations in mtDNA (Table 3). Out of a total 44 mtDNA alterations observed in RTS patient cells, 30 were observed in L9552914-J, indicating that loss of both p53 and RECQL4 function enhanced the rate of mtDNA alterations 5-6 fold. Fifteen mutations were located in the non-coding mtDNA, the displacement loop (D-loop), known to be involved in the control of both mitochondrial replication and transcription (Clayton, 1982). In two of the three RTS patients, insertion mutations were present in a polycytidine stretch (C-tract or D310), described as a hot spot for mutations in many cancers (Sanchez-Cespedes et al., 2001) and involved in mtDNA replication and transcription (Ghivizzani et al., 1994). Out of the rest 29 alterations, 6 were non-synonymous coding region mutations (i.e. resulting in an amino-acid change in genes coding for OXPHOS enzymes), 15 were synonymous, 3 were in t-RNAs, 4 in 12/16S RNAs and one was a heteroplasmic mutation. Susceptibility of the mitochondrial coding region to DNA alterations provide an explanation regarding the perturbation in electron transport chain leading to alteration in mitochondrial potential, and generation of oxidative stress. A vast majority of mutations in RTS cells were homoplasmic in nature, supporting earlier reports of high frequency of homoplasmic mutations detected in human tumors (Mambo et al., 2003).

TABLE 3
Mutations detected in the mitochondrial DNA of RECQL4 fibroblasts {reference revised Cambridge Reference
Sequence (rCRS)}

| Cells | Nucleotide change | Position of mutation | Type of mutation |
|---|---|---|---|
| AG03587 | 146T > C | D-loop | Non-coding mtDNA |
| | 195T > C | D-loop | Non-coding mtDNA |
| | 309_310insCT | D-loop | Non-coding mtDNA |
| | 310T > C | D-loop | Non-coding mtDNA |
| | 5471G > A | MT-ND2 | Synonymous mutation in NADH dehydrogenase 2 |
| | 5492T > C | MT-ND2 | Synonymous mutation in NADH dehydrogenase 2 |
| | 9145G > R | MT-ATP6 | Heteroplasmic mutation in ATP synthase 6 |
| | 12010C > T | MT-ND4 | Synonymous mutation in NADH dehydrogenase 4 |
| | 14497A > G | MT-ND6 | Synonymous mutation in NADH dehydrogenase 6 |
| B1865425K | 152T > C | D-loop | Non-coding mtDNA |
| | 4336T > C | MT-TQ | Mutation in tRNA-glutamine |
| | 5319A > G | MT-ND2 | Non-synonymous mutation (Thr to Ala) in NADH dehydrogenase 2 |
| | 8563A > G | MT-ATP6 | Non-synonymous mutation (Thr to Ala) in ATP synthase 6 |
| | | MT-ATP8 | Synonymous mutation in ATP synthase 6 |
| L9552914-J | 73A > G | D-loop | Non-coding mtDNA |
| | 150C > T | D-loop | Non-coding mtDNA |
| | 310T > C | D-loop | Non-coding mtDNA |
| | 311-312insTCC | D-loop | Non-coding mtDNA |
| | 709G > A | MT-RNR1 | Mutation in 12S RNA |
| | 1888G > A | MT-RNR2 | Mutation in 16S RNA |
| L6474163-L (cont.) | 2302A > G | MT-RNR2 | Mutation in 16S RNA |
| | 2706A > G | MT-RNR2 | Mutation in 16S RNA |
| | 4216T > C | MT-ND1 | Non-synonymous mutation (Tyr to His) in NADH dehydrogenase 1 |
| | 4917A > G | MT-ND2 | Non-synonymous mutation (Asn to Asp) in NADH dehydrogenase 2 |
| | 7028C > T | MT-COI | Synonymous mutation in cytochrome c oxidase I |
| | 8697G > A | MT-ATP6 | Synonymous mutation in ATP synthase 6 |
| | 10463T > C | MT-TR | Mutation in tRNA arginine |
| | 11251A > G | MT-ND4 | Synonymous mutation in NADH dehydrogenase 4 |
| | 11719G > A | MT-ND4 | Synonymous mutation in NADH dehydrogenase 4 |
| | 11812A > G | MT-ND4 | Synonymous mutation in NADH dehydrogenase 4 |
| | 13368G > A | MT-ND5 | Synonymous mutation in NADH dehydrogenase 5 |
| | 14233A > G | MT-ND6 | Synonymous mutation in NADH dehydrogenase 6 |
| | 14766C > T | MT-CYB | Non-synonymous mutation (Thr to Ile) in cytochrome b |
| | 14905G > A | MT-CYB | Synonymous mutation in cytochrome b |
| | 15452C > A | MT-CYB | Non-synonymous mutation (Leu to Ile) in cytochrome b |
| | 15499C > T | MT-CYB | Synonymous mutation in cytochrome b |
| | 15607A > G | MT-CYB | Synonymous mutation in cytochrome b |
| | 15928G > A | MT-TT | Mutation in tRNA threonine |
| | 16114C > T | D-loop | Non-coding mtDNA |
| | 16126T > C | D-loop | Non-coding mtDNA |
| | 16153G > A | D-loop | Non-coding mtDNA |
| | 16192C > T | D-loop | Non-coding mtDNA |
| | 16294C > T | D-loop | Non-coding mtDNA |
| | 16519T > C | D-loop | Non-coding mtDNA |

### Example 8

Loss of Both p53 and RECQL4 Led to Increased mtDNA Mutations and Enhanced Metastatic Potential of RTS Fibroblasts.

Next we wanted to determine whether enhanced mtDNA mutations in RTS patients can be correlated to the functional status of RECQL4 and p53 in RTS patient cells. Hence we studied the relative metastatic potential of NHF and RTS cells using BD BioCoat™ Matrigel™ Invasion Chamber. 106 cells/cm2 was seeded in 24-well chamber and incubated for 22 h at 370C. The non-invading cells were removed by scrubbing, the cells which have migrated to the lower surface of the membrane were fixed by 100% methanol, stained with 1% Toluidine blue, photographed at 63X in Upright Axioimager M1 motorized Epifluorescence microscope equipped with a high resolution AxioCam MRm Rev. 2 camera. The experiment was done in triplicate for three times and data represented is combined from all datasets. HCT116, used as a positive control, demonstrated robust invasion through the basement membrane. While NHF showed negligible invasive capability, RTS fibroblasts invaded the basement membrane, but to varied extent (FIG. 11C). The extent of invasion of L9552914-J was even greater than HCT116 and was about 10-fold more than AG03587 and B1865425K. The relative invasive capabilities of the tested cells mirrored the number of mtDNA mutations, thereby indicating that mitochondrial deregulation in RTS patients probably plays a very important role during cancer development.

### Advantages of the Inventions

This work provides an insight into the potential diverse mitochondrial functions of RECQL4. Further, it unfolds the functions of RECQL4 which may be involved in increasing fidelity of the mitochondrial replication process and/or regulating the diverse processes that control the Reactive Oxygen Species (ROS) level.

These findings indicate that RECQL4/RECQL4 variant-p53 adduct can be a target for development of vaccines, and as a first generation vaccine candidate, GFP-tagged RECQL4 can be cloned into lentivirus vector, transduced into RTS patient cells and constitutively expressed. The “rescued” patient cells can be scored for the mitochondrial functions.

For a second generation vaccine candidate, an exogenous mitochondrial import leader peptide can be attached to the GFP-tagged RECQL4 so that the mitochondrial localization of the helicase is faster and complete. This modified full-length RECQL4 with the exogenous mitochondrial leader peptide can be constitutively expressed using the lentivirus system.

For a third generation vaccine candidate, modifications of the wildtype GFP-tagged RECQL4 can be done that a minimum amount of the helicase is expressed for the maximum efficacy. Recombinant GFP-RECQL4 construct can be generated which will contain the mitochondrial localization signal (either its own or an exogenous peptide), the minimum domain which would allow RECQL4 to bind to p53 and the helicase domain through which RECQL4 probably affects mitochondrial replication. These GFP-tagged constructs can be cloned into the lentivirus expression system, expressed into patient cells and scored for mitochondrial functions.

We claim:

1. A method of identification of functional RECQL4/RECQL4 variant -p53 complex wherein the said method comprising the steps of:
a) interaction of p53 and RECQL4/RECQL4 variant
b) import of RECQL4/RECQL4 variant and p53 complex through mitochondrial Tom 40 receptor complex.
c) import of RECQL4/RECQL4 variant and p53 complex into mitochondria through Tom 40 receptor complex via the MLS of RECQL4.
d) the localization of the p53-RECQL4/RECQL4 variant complex in the mitochondrial nucleoids and;
measurement of ROS, and identification of mitochondrial mutations in Rothmund-Thomson Syndrome.

2. A method as claimed in claim 1, wherein the interaction of RECQL4 and p53 is through the nuclear localization signal of both RECQL4 and p53.

3. A method as claimed in claim 1, wherein the import of RECQL4/RECQL4 variant-p53 into mitochondria is through Tom 40 receptor complex.

4. A method as claimed in claim 1, wherein the import of RECQL4/RECQL4 variant-p53 into Tom 40 receptor complex is through the MLS of RECQL4.

5. A method as claimed in claim 4, wherein the MLS of RECQL4 is located at the N-terminal region of RECQL4.

6. A method as claimed in claim 5, wherein MLS of RECQL4 is located between the 13th and 18th peptide of the N-terminal region of RECQL4.

7. A method as claimed in claim 1, wherein mitochondrial mutations identifies RTS.

8. A method as claimed in claim 1, wherein ascorbic acid is an ROS scanvenger in Rothmund-Thomson Syndrome.
